(19) United States
(12) Patent Application Publication
(10) Pub. No.: US 2021/0316247 A1
(43) Pub. Date: Oct. 14, 2021

(54) RELEASE KIT INCLUDING CARRIER CAPABLE OF ADSORBING HIGH-CAPACITY CHLORINE DIOXIDE GAS AND PREPARATION APPARATUS CAPABLE OF PREPARING CARRIER

(71) Applicant: REnBiz, Pohang-si (KR)

(72) Inventor: Byoung Eog KIM, Pohang-si (KR)

(21) Appl. No.: 17/184,788

(22) Filed: Feb. 25, 2021

(30) Foreign Application Priority Data
Apr. 10, 2020 (KR) 10-2020-0044212

(51) Int. Cl. B01D 53/04; B01J 20/20; B01J 20/28; B01J 20/32; B01J 20/30

(52) U.S. Cl. CPC B01D 53/0446; B01J 20/20; B01J 20/28059; B01J 20/3204; B01D 2257/2025; B01J 20/3092; B01J 20/28019; B01J 20/28061; B01J 20/3028

(57) ABSTRACT

The present disclosure relates to a carrier with high capacity which is capable of adsorbing a chlorine dioxide gas at high concentration and a method of preparing a release kit which is capable of releasing the chlorine dioxide gas at a certain concentration for a long period of time, and more particularly, provides a preparation method of a carrier which is capable of adsorbing a high concentration chlorine dioxide gas at high capacity and capable of maintaining a physically and chemically stable state for a long period of time, the carrier including a silica gel, i.e., an indicator material which may indicate whether or not the chlorine dioxide gas is adsorbed or desorbed, by colors; and the present disclosure provides a method capable of manufacturing a chlorine dioxide gas sterilization device which consists of a release kit enabling the chlorine dioxide gas of a certain concentration to be continuously released from the carrier for a long period of time, an internal configuration of the kit including a well-light shielded sealed container capable of storing the carrier and the indicator, an inner upper part of the sealed container containing an aromatic gel that is capable of suppressing a distinctive smell of chlorine dioxide and immersed in a super absorbent polymer, which is configured so that the release amount and the release duration can be adjusted when adjusting the hole size by forming a hole with a predetermined size in a lid of the sealed container so that the chlorine dioxide gas can be released at a certain concentration from the inside of the sealed container to the outside thereof for a long period of time, and which not only can be subminiaturized by allowing the configuration of the device to become structurally very simple when applying the kit to the sterilization device, but also is more safe and inexpensive by excluding the use of harmful chemical substances, i.e., raw materials required for generating the chlorine dioxide gas. Further, as a carrier according to the present disclosure may be reusable up to five times, the carrier has great advantages in terms of recycling of resources and maintenance costs.

### CROSS-REFERENCE TO RELATED APPLICATIONS

This application claims the priority of Korean Patent Application No. 10-2020-0044212 filed on Apr. 10, 2020, in the Korean Intellectual Property Office, the disclosure of which is incorporated herein by reference.

### BACKGROUND

### Field

The present disclosure relates to a carrier which is capable of adsorbing a high concentration chlorine dioxide gas at high capacity and a method of preparing a kit which is capable of releasing the chlorine dioxide gas at a certain concentration for a long period of time, and more particularly, to a carrier which is capable of adsorbing a high concentration chlorine dioxide gas at high capacity and capable of being maintained physically and chemically stably even for several months, and a method of preparing a kit which is capable of continuously releasing the chlorine dioxide gas at a certain concentration for a long period of time by using the carrier.

### Related Art

As infection problems caused by pathogenic bacteria such as MERS, coronavirus, and the like frequently occur, social interest in sterilization techniques has recently been increasing in our society. The majority of the sterilization techniques include sterilization techniques which are based on physical energies such as UV, ultrasonic waves, etc., or sterilization techniques which are based on chemical substances such as sterilizing disinfectants, and sterilization techniques using chemical substances such as chlorine dioxide in terms of effectiveness and economic feasibility, while enabling a wide range of wide-area sterilization among the majority of the sterilization techniques have been continuously developed.

In general, chlorine dioxide has been known as an environment-friendly sterilizing disinfectant which has strong oxidizing power, sterilizing disinfection power, and deodorizing power, does not produce carcinogenic organic matters such as trihalomethanes (THMs), haloacetic acids (HAAs), and haloacetonitriles (HANs) unlike other chlorine disinfecting sterilizers, does not react with other organic substances to produce organic chlorine compounds, and is quickly decomposed by sunlight or temperature so that chlorine dioxide does not remain. These highly selective properties of chlorine dioxide are due to its sterilizing action harmless to the human body. That is, chlorine dioxide does not produce harmful sterilizing by-products such as trihalomethanes and polychlorobiphenyls (PCBs).

In order to prepare a chlorine sterilizing substance such as chlorine dioxide using chemical reactions, a chlorine supply material that provides chlorine and a decomposition source that can decompose the chlorine supply material are required together, and, in this regard, various compounds such as chlorine, hypochlorite, chlorous acid, and the like as the chlorine supply material have been used in the related art. Furthermore, acidic substances such as hydrochloric acid and the like and compounds such as ozone and the like have been used as a decomposition source for producing chlorine dioxide by decomposing the chlorine supply material, or energy means such as ultraviolet rays and the like have been used.

For example, although sodium chlorite or sodium hypochlorite is used as a chlorine supply source, and an acid substance such as citric acid, acetic acid, lactic acid, or the like, hydrochloric acid, or ozone is used as an oxidizer in Korean Patent No. 10-1416785 and Korean Patent No. 10-770222, and ultraviolet rays are used as a decomposition source in Korean Patent Laid-Open Publication No. 10-2005-0015949 and Korean Patent No. 10-1806283 that are other related arts, strong corrosive materials such as acidic substances are diluted and used as the decomposition source, or mechanical means such as ultraviolet rays are used in all of the related arts. Therefore, there have been problems that an apparatus becomes complicated, and the size of the apparatus is increased. In addition, as a high-priced generating system must be established in order to continuously utilize the chlorine dioxide gas in sterilization since a chlorine dioxide gas cannot be stored for a long time, sterilization is mainly done for pretreatment purposes before the circulation of agricultural and marine products or foods. This sterilizing pretreatment has the disadvantage of not continuously generating the chlorine dioxide gas and thus being unable to perform a sterilizing process. Accordingly, in order to use the chlorine dioxide gas in various places that require sterilization, it is necessary to develop techniques that can generate the chlorine dioxide gas even without a chlorine supply source and a decomposer being mounted on the generating system.

There are Korean Patent Nos. 10-1443455 and 10-2008823 as related arts that do not require a chlorine dioxide generation system.

These patented techniques have been developed into techniques which provide a chlorine dioxide gas release pack (sachet) using silica-gel or may generate chlorine dioxide stably and continuously as a method for continuous treatment of the chlorine dioxide gas in circulation.

However, although the invention 10-1443455 claims that it may adjust the release concentration and duration of the chlorine dioxide gas by varying the amount of silica gel adsorbed with the chlorine dioxide gas, the type of a packing film, and the storage temperature conditions depending on the purpose of use, and particularly, it not only increases the freshness and quality of agri-foods, but also may contribute to securing storage stability and microbial safety during circulation by enabling the chlorine dioxide gas to be continuously released, it has problems in terms of human safety in handling as the duration time is only about 7 days, and the first-day initial discharge concentration is very high. Furthermore, the manufacturing process is complicated due to the need to adjust the permeability of the film to increase prolonged persistence. Moreover, since the total adsorption amount that is finally adsorbed is insufficient as the concentration of the chlorine dioxide gas used in the adsorption is only 2,000 ppm and the adsorption capacity of an adsorbent is also low, it has limitations in maintaining the release concentrations for a long period of time.

Similarly, the invention 10-2008823 is a technique of providing sustained release chlorine dioxide generating formulation and generating pack including a first adsorbent onto which chlorite coated with a polymer material is adsorbed and an activator which reacts with chlorite adsorbed onto the first adsorbent to generate chlorine dioxide, in which chemicals for generating chlorine dioxide such as raw materials, the activator, etc. are contained in the product. Furthermore, it has problems that separate moisture for generating chlorine dioxide is required, the initial release amount is very high, and the release amount is rapidly decreased to a 1/40 level after two hours when the generating pack is not packed with a porous film, and the release amount has a severe deviation for 47 days even when the porous film is used.

### RELATED ART DOCUMENT

### Patent Documents

(Patent Document 1) Korean Patent No. 10-1416785

(Patent Document 2) Korean Patent No. 10-770222

(Patent Document 3) Korean Patent Laid-Open Publication No. 10-2005-0015949

(Patent Document 4) Korean Patent No. 10-1806283

(Patent Document 5) Korean Patent No. 10-1443455

(Patent Document 6) Korean Patent No. 10-2008823

### SUMMARY

The present disclosure is intended to improve problems including 1) short persistence in chlorine dioxide release, 2) a fact that long-term release of chlorine dioxide at a certain concentration is not maintained, and 3) complexity of preparation process and 4) the use of harmful chemicals depending on the use of the packing film, i.e., problems of the related art. An object of the present disclosure is to provide a method of preparing a new carrier capable of physically and chemically stably adsorbing and maintaining a high concentration chlorine dioxide gas at high capacity for a long period of time and a method of preparing a kit capable of continuously releasing the chlorine dioxide gas at a certain concentration for a long period of time from such a carrier, unlike in the case of the invention 10-1443455 in which it is difficult to release chlorine dioxide and maintain a certain concentration of chlorine dioxide for a long period of time by adsorbing a low concentration chlorine dioxide gas onto the carrier.

Furthermore, another object of the present disclosure is to provide a method of preparing a carrier onto which high concentration chlorine dioxide is adsorbed by manufacturing a separate apparatus capable of generating a high concentration chlorine dioxide gas and continuously adsorbing onto the carrier in a safe and well-ventilated environment. The present disclosure provides a method of preparing a carrier onto which only a pure chlorine dioxide gas free from harmful chemical substances such as separate raw material, decomposer, and the like required in the production of the chlorine dioxide gas is adsorbed unlike the prior art 10-2008823 by allowing configuration of the apparatus to prepare the carrier through a series of generation and adsorption devices including a reaction tank that produces chlorine dioxide and an adsorption bed capable of adsorbing chlorine dioxide onto the carrier.

Furthermore, a release kit prepared by a method according to the present disclosure includes a carrier, a well-light shielded sealed container capable of containing a bead-type aromatic gel that eliminates distinctive smells of an indicator and chlorine dioxide, and a container lid. Therefore, there is an advantage that the kit is prepared very easily and inexpensively, and a certain concentration chlorine dioxide gas may be released for a long period of time by easily adjusting the release amount and release period depending on the size of the hole in the container lid.

In addition, a carrier according to the present disclosure also has a very large advantage in terms of resource recycling since the carrier may be reused about five times after use.

Meanwhile, technical tasks to be accomplished in the present disclosure are not limited to the technical tasks mentioned above, and other technical tasks which have not been mentioned will be clearly understood by a person with ordinary skill in the art to which the present disclosure pertains from the following description.

The present disclosure provides a chlorine dioxide release kit including: a carrier onto which a chlorine dioxide gas is adsorbed; a sealed container; and a lid, in which the carrier onto which the chlorine dioxide gas is adsorbed is prepared by mixing a powder having composition ratios of 50 wt % to 69 wt % of SiO₂, 10 wt % to 15 wt % of Al₂O₃, 5 wt % to 10 wt % of Fe₂O₃, 5 wt % to 10 wt % of MgO, 3 wt % to 5 wt % of CaO, and 4 wt % to 8 wt % of others including TiO₂, K₂O, and SO₃ with an activated carbon powder.

Further, the present disclosure provides a chlorine dioxide release kit in which the activated carbon powder has a composition ratio of 10 wt % to 50 wt %.

Further, the present disclosure provides a chlorine dioxide release kit in which the carrier onto which the chlorine dioxide gas is adsorbed is made of spherical beads having an average size of 2 mm to 3 mm.

Further, the present disclosure provides a chlorine dioxide release kit in which the carrier onto which the chlorine dioxide gas is adsorbed has a specific surface area (BET) distribution of about 70 m²/g to 150 m²/g.

Further, the present disclosure provides a chlorine dioxide release kit in which the lid has a hole with a diameter of 1 mm to 3 mm to determine the release amount and the duration time.

Further, the present disclosure provides a chlorine dioxide release kit further including a polyamide air freshener gel for preventing a unique smell of chlorine dioxide from being generated from the chlorine dioxide release kit.

Further, the present disclosure provides a chlorine dioxide release kit in which the carrier onto which the chlorine dioxide gas is adsorbed further includes a silica gel, i.e., an indicator material which can indicate whether or not the chlorine dioxide gas is adsorbed or desorbed by colors.

Furthermore, the present disclosure provides a preparation apparatus capable of preparing a carrier onto which a chlorine dioxide gas is adsorbed, the preparation apparatus including: a reaction tank capable of producing aqueous chlorine dioxide by dissolving a solid NaClO₂ powder, i.e., a raw material of chlorine dioxide in water, and adding acid to the solid NaClO₂ powder-dissolved water; a gas pump capable of discharging a chlorine dioxide gas to the outside by injecting air into the reaction tank in a flow amount of 2 L/min or more; an adsorption bed capable of filling the carrier and adsorbing the chlorine dioxide gas; and a silica gel, i.e., an indicator capable of indicating the adsorption amount and the adsorption progress degree of the chlorine dioxide gas by colors.

Further, the present disclosure provides a preparation apparatus capable of preparing a carrier onto which a chlorine dioxide gas is adsorbed, the preparation apparatus further including any one or more among a stirrer, a thermometer, a pressure gauge, a flowmeter, a cryostat, and a granulated activated carbon bed capable of removing the portion of the chlorine dioxide gas when a portion of the chlorine dioxide gas is discharged to the outside after the adsorption process is completed.

The present disclosure relates to a method of preparing a high-capacity carrier capable of adsorbing a high concentration chlorine dioxide gas and a method of preparing a release kit for enabling the carrier to be used in devices for various sterilization purposes.

When a chlorine dioxide sterilization device is manufactured by means of the present disclosure, the present disclosure has advantages of enabling the device to be subminiaturized and allowing the configuration of the device to become structurally very simple. As use and handling of reaction raw materials, i.e., harmful chemicals necessary for the generation of a chlorine dioxide gas are not required, and it is possible to release chlorine dioxide with a certain concentration continuously, the health of the human body may be secured from various bacteria and viruses in a safe environment without harm to the human body. Further, it may be possible to relieve the discomfort that people who are sensitive to smell may feel when used in indoor spaces by applying a bead-type aromatic gel to the release kit, thereby suppressing a distinctive smell of chlorine dioxide. Further, as the carrier obtained through the present disclosure has been shown to be reusable about five times even after use in the adsorption of chlorine dioxide with a high concentration, the carrier has great advantages in terms of recycling of resources and maintenance costs.

### BRIEF DESCRIPTION OF THE DRAWINGS

The significance of features and advantages of the present disclosure will be better understood by referring to the accompanying drawings. However, it should be understood that the drawings are intended solely for illustrative purposes and do not define the limitations of the present disclosure.

FIG. 1 shows an apparatus which generates a high concentration chlorine dioxide gas and adsorbs it onto a carrier.

FIGS. 2A and 2B show an analyzer which may measure the concentration of chlorine dioxide contained in the carrier in an aqueous solution or in the air after the adsorption according to the present disclosure.

FIGS. 3A and 3B show a device for measuring the release amount of chlorine dioxide in a sealed container (40 L).

FIG. 4 quotes a graph showing the release concentration of silica gel indicated in the invention 10-1443455 over time.

FIGS. 5A and 5B shows photograph taken before and after adsorbing a high concentration chlorine dioxide gas onto the carrier, in which a left photograph is a photograph taken before the reaction, and a right photograph is a photograph taken after the reaction.

FIGS. 6A and 6B show a device capable of measuring the release amount and the duration time of chlorine dioxide for a long period of time.

### DETAILED DESCRIPTION

Hereinafter, embodiments of the present disclosure will be described in detail with reference to the accompanying drawings so that one of ordinary skill in the art to which the present disclosure pertains will easily be able to implement the present disclosure. However, as the description of the present disclosure is only embodiments for structural or functional explanations, the right scope of the present disclosure shall not be construed as restricted by the embodiments described herein. That is, as the embodiments can be variously changed and may have various shapes, the right scope of the present disclosure should be understood to include equivalents that can realize technical ideas. Further, as the purposes or effects presented in the present disclosure do not mean that a particular embodiment should include all of them or only those effects, the right scope of the present disclosure should not be understood to be limited thereby.

The meaning of the terms described in the present disclosure should be understood as follows.

The terms “first”, “second”, etc. are intended to distinguish one constituent element from another, and the scope of rights should not be limited by these terms. For example, a first constituent element may be named as a second constituent element, and, similarly, the second constituent element may also be named as the first constituent element.

Although the constituent element may be directly connected to the other constituent element when it is mentioned that any constituent element is “connected” to another constituent element, it should be understood that another constituent element may exist therebetween. On the other hand, when any constituent element is mentioned to be “directly connected” to another constituent element, it should be understood that another constituent element does not exist therebetween. Meanwhile, different expressions that describe the relationship between the constituent elements, i.e., “between” and “right between” or “neighboring to” and “directly neighbor to”, etc. should also be interpreted in the same manner.

An expression of the singular number should be understood to include an expression of the plural number unless clearly defined otherwise in the context. In the present specification, it should be understood that a term such as “comprises” or “having” is used to specify existence of a feature, a number, a step, an operation, a constituent element, a part, or a combination thereof described in the specification, but it does not preclude the possibility of the existence or addition of one or more other features, numbers, steps, operations, constituent elements, parts, or combinations thereof.

Unless otherwise defined, all terms used herein have the same meaning as commonly understood by one of ordinary skill in the art to which the present disclosure pertains. Terms, such as those defined in commonly used dictionaries, should be interpreted as having meanings that are consistent with those in the context of the related art, but are not interpreted as having ideal or excessively formal meanings unless clearly defined in the present disclosure.

Hereinafter, specific contents for achieving the present disclosure are as follows:

### Preparation Method of Carrier

First, a method of preparing a carrier with a capacity enabling a chlorine dioxide gas to be adsorbed at a high concentration is as follows.

A powder with component composition ratios in the same ranges as in the table below was obtained by mixing powders (an average particle size of 10 μm) of various minerals (bentonite, zeolite, meerschaum, and alumina) large amounts of which were present in nature.

### Composition Ratios of Minerals Forming the Carrier

TABLE 1

| Chemical | Contents | Chemical | Contents |
|---|---|---|---|
| components | (wt %) | components | (wt %) |
| SiO₂ | 50~69 | MgO | 5~10 |
| Al₂O₃ | 10~15 | CaO | 3~5 |
| Fe₂O₃ | 5~10 | Others (TiO₂, K₂O, SO₃) | 8~12 |

The powder was uniformly mixed with an activated carbon powder with a specific surface area so that the mineral powders were well dispersed on the surface of the activated carbon. Although the activated carbon powder used here also acts as a binder required for making the mineral powders into spherical pellets, the mixed powders interfered with the adsorption capacity of a high concentration chlorine dioxide gas during adsorption when large amounts of the mineral powders were mixed with the activated carbon powder. Therefore, the mineral powders were mixed at a ratio of 10 to 50 wt % in the present disclosure. Preferably, the activated carbon powder contained in the mixed powders was used in an amount of 15 to 25 wt %.

Such a mixed powder was prepared into spherical beads with an average size of 2 mm to 3 mm by using a tableting machine which had been widely used in the pharmaceutical industry. The prepared spherical bead carrier showed a specific surface area (BET) distribution of about 70 m²/g to 150 m²/g depending on the mixed amount of activated carbon, and the carrier was used after sufficiently drying the carrier under vacuum conditions before using the carrier in the adsorption.

Apparatus for Adsorbing Chlorine Dioxide onto Prepared Carrier

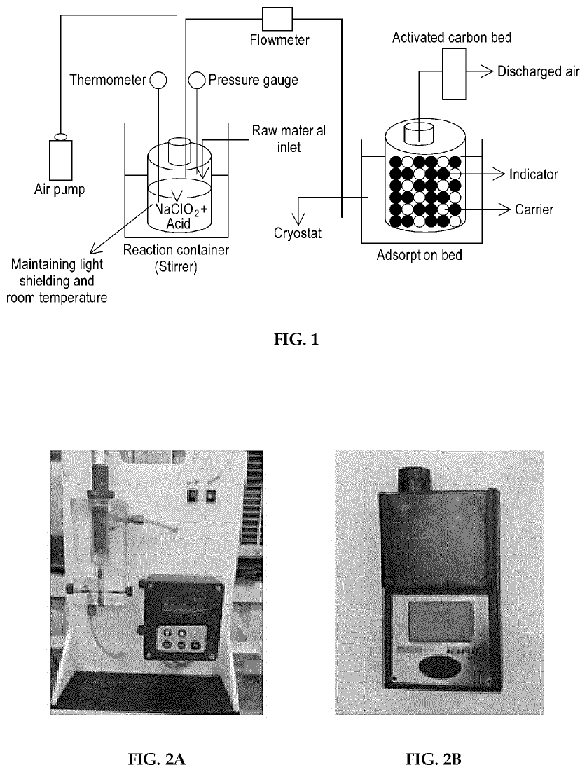

The prepared bead carrier adsorbed a high concentration chlorine dioxide gas through an apparatus as shown in FIG. 1. The configuration of the apparatus was included of: a reaction tank capable of preparing aqueous chlorine dioxide by dissolving a solid NaClO₂ powder, i.e., a raw material in water and adding acid to the solid NaClO₂ powder-dissolved water; a gas pump capable of discharging the chlorine dioxide gas to the outside by injecting air into the reaction tank in a flow amount of 2 L/min or more; an adsorption bed capable of filling the carrier and adsorbing the chlorine dioxide gas; and a silica gel, i.e., an indicator capable of indicating the adsorption amount and the adsorption progress degree of the chlorine dioxide gas by colors. In addition, the configuration of the apparatus was included of a stirrer, a thermometer, a pressure gauge, a flowmeter, a cryostat, and a granulated activated carbon bed capable of removing the portion of the chlorine dioxide gas when discharging a portion of the chlorine dioxide gas to the outside after completing the adsorption process (referred to FIG. 1).

### Evaluating Adsorption Capacity of Prepared Carrier

After comparing a chlorine dioxide adsorption capacity of the carrier prepared through the above-mentioned processes with that of a silica gel which had been widely used as a common adsorbent in the related art, comparison results were evaluated. An analyzer used in the evaluation process included equipment for measuring chlorine dioxide concentration in water (FIGS. 2A and 2B), which was manufactured by Reiss GmbH in Germany. The evaluation method included putting 1 g of the carrier and 1 g of the silica gel, i.e., an indicator, into a brown reagent bottle filled with 1 L of distilled water, sealing the brown reagent bottle, repeatedly measuring the concentration of chlorine dioxide in water at intervals of ten minutes, and measuring the maximum concentration value with the lapse of 30 minutes as a result. Further, as results of measuring changes in adsorption capacity in the same manner as above, a carrier obtained by repeating adsorption and desorption of a chlorine dioxide gas five times also showed a decrease in adsorption capacity of about 10%, and these results are aggregated and presented in Table 2 below.

### Adsorption Capacity Analysis Results of a Chlorine Dioxide-Adsorbed Carrier

TABLE 2

| | After 10 | | After 20 | After 30 | After 1 |
|---|---|---|---|---|---|
| Component | minutes | | minutes | minutes | hour |
| Using the | 18~20 | ppm | 30~35 ppm | 50~57 ppm | ~50 ppm |
| carrier once | | | | | |
| Reusing the | 16~18 | ppm | 26~30 ppm | 45~48 ppm | ~45 ppm |
| carrier 5 times | | | | | |
| Indicator | 8~12 | ppm | 15~20 ppm | 25~30 ppm | ~30 ppm |

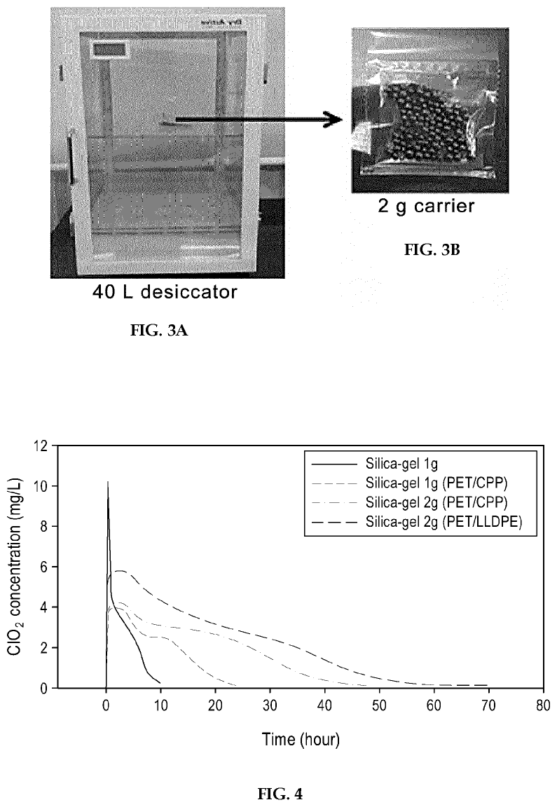

Further, after sealing 2 g of a chlorine dioxide-adsorbed carrier containing an indicator with a medicine wrapper formed of transparent polyethylene in a 40 L capacity desiccator as shown in FIGS. 3A and 3B, and repacking the indicator-containing chlorine dioxide-adsorbed carrier sealed with the medicine wrapper with an aluminum foil to minimize the decomposition impact due to light, there was no change in the release concentration by the end of five days, and there was a decrease phenomenon of the release concentration at the end of the six days, as results of examining changes in the concentration with the passage of time using a chlorine dioxide gas measuring instrument. The results are shown in Table 3 below (referred to FIGS. 3A and 3B)

### Results of Examining Release Amounts of the Chlorine Dioxide-Adsorbed Carrier in a Confined Space

TABLE 3

| | | | | | | | | |
|---|---|---|---|---|---|---|---|---|
| Component | 1 | day | 2 | days | 3 | days | 4 | days |
| Carrier + | 1.8~2.0 | ppm | 1.8~2.0 | ppm | 1.8~2.0 | ppm | 1.8~2.0 | ppm |
| indicator | 5 | days | 6 | days | 7 | days | 8 | days |
| (2 g) | 1.8~2.0 | ppm | 1.6~1.8 | ppm | 1.3~1.5 | ppm | 1.0~1.4 | ppm |

When comparing the results in Table 3 above with those of the conventional invention in FIG. 4 below, it can be seen that the adsorption capacity of chlorine dioxide in the carrier obtained through the present disclosure is very high.

### Method of Preparing Chlorine Dioxide Release Kit

A release kit was prepared so that a chlorine dioxide-adsorbed bead type carrier could release chlorine dioxide at a certain concentration for a long period of time. First, 400 g of a high concentration chlorine dioxide gas-adsorbed carrier obtained through the above-mentioned processes of FIG. 1 was put into a 500 ml capacity narrow mouth-type sealed container which was made of polyethylene and well-light shielded. An air freshener-containing polyacrylamide gel, i.e., a super absorbent polymer was put into an upper portion of the carrier in an amount of 20 g to 50 g. A hole with a size of 1 mm to 5 mm was formed in a lid of the sealed container using a drill. Due to the hole size determining the release amount and the duration time, a hole size of preferably 1 mm to 3 mm was shown to be optimal. The reason for this was that it was difficult to maintain a certain concentration since the release amount increased, but the duration time became shorter if the hole size was larger than 5 mm, and, conversely, the duration time became longer, but the release amount decreased if the hole size was smaller than 1 mm. As results of periodically measuring the concentration of chlorine dioxide released from this prepared release kit at room temperature over three months, the following results were obtained. After checking that 12 to 15 ppm of a chlorine dioxide gas, i.e., a 60 to 70% level compared to the initial 3 months, was released at the end of six months later as the release concentration gradually decreased after the lapse of 3 months in case of the hole size of 2 mm, the results are summarized in Table 4 below.

### Release Concentrations Depending on Hole Sizes and Time Elapses of the Sealed Container

TABLE 4

| Hole size | 0 month | 1 month | 2 months | 3 months | | ~6 months | |
|---|---|---|---|---|---|---|---|
| 1 mm | 15~20 ppm | 15~20 ppm | 15~20 ppm | 15~20 | ppm | 14~6 | ppm |
| 2 mm | 20~25 ppm | 20~25 ppm | 20~25 ppm | 20~25 | ppm | 12~5 | ppm |
| 3 mm | 24~28 ppm | 24~28 ppm | 24~28 ppm | 15~20 | ppm | 6~10 | ppm |
| 4 mm | 30~35 ppm | 30~35 ppm | 26~30 ppm | 12~16 | ppm | 4~8 | ppm |
| 5 mm | 40~45 ppm | 40~45 ppm | 20~25 ppm | 6~10 | ppm | 1~2 | ppm |

### Example 1

### Experimental Example 1

A powder with a component composition including 56 wt % of SiO₂, 13 wt % of Al₂O₂, 8 wt % of Fe₂O₃, 8 wt % of MgO, 4 wt % of CaO, 3 wt % of SO₂, 2 wt % of TiO₂, 2 wt % of K₂O, and 4 wt % of others was obtained by mixing minerals easily available in nature, e.g., powders (an average particle size of 10 μm) of bentonite, zeolite, meerschaum, alumina, etc.

After mixing an activated carbon powder with this obtained powder at a weight ratio of 20% (a ratio of 1:4) to act as a binder and to increase the non-surface area, the mixture was homogenized so that mineral powders were well dispersed on the surface of activated carbon. The finally mixed powder was prepared into spherical beads with an average size of 2 to 3 mm by using a tableting machine which had been widely used in the pharmaceutical industry so as to secure proper strength and air permeability of the adsorption process. The prepared spherical bead carrier showed a specific surface area (BET) range of about 80 m²/g to 150 m²/g depending on the ratio of the powder mixed with activated carbon, and the carrier was sufficiently dried under vacuum conditions of 100° C. or less before using the carrier in the chlorine dioxide gas adsorption. The progress situation of adsorption was enabled to be checked by mixing the drying process-completed carrier with a silica gel which could be used as an indicator by having a yellowish disposition when adsorbing chlorine dioxide onto the silica gel.

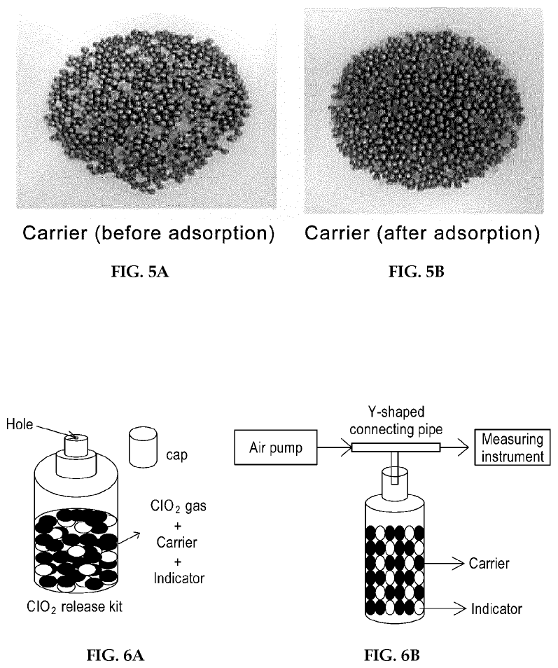

The adsorption of chlorine dioxide gas was carried out by manufacturing an adsorption apparatus as shown in FIG. 1. After generating a high concentration chlorine dioxide gas by reacting acid (hydrochloric acid, various organic acids, etc.), i.e., a chlorine dioxide-producing decomposer, with NaClO₂ in an aqueous solution state, i.e., a raw material, in the reaction tank, the generated high concentration chlorine dioxide gas was adsorbed onto the carrier in an adsorption bed maintaining a low temperature (11° C. or less). Photographs taken before and after adsorption of the adsorption process-completed carrier are shown in FIGS. 5A and 5B. As results of putting 1 g of the obtained carrier into a light-shielded 1 L capacity brown reagent bottle filled with 1 L of distilled water, sealing the brown reagent bottle, and measuring the concentration of chlorine dioxide dissolved in water after 30 minutes, a concentration of about 57.1 ppm was measured.

Further, after wrapping 2 g of a chlorine dioxide-adsorbed carrier with a transparent medicine wrapper, and light-shielding the exterior of the carrier wrapped with the transparent medicine wrapper with an aluminum foil, the release concentration was examined in a 40 L desiccator for 8 days (referred to FIGS. 2A and 2B). As a result, the concentration was shown to be constantly maintained to a 2.0 ppm level until the fifth day, and then the concentration was gradually decreased from the sixth day to show that chlorine dioxide was released in an amount of about 1.4 ppm by reducing the release amount on the eighth day (referred to FIGS. 5A and 5B).

### Experimental Examples 2 to 13

Experiments were conducted on respective composition ratios of Experimental Examples 2 to 13 by varying only the composition ratio of each mineral in the same manner as in Experimental Example 1 above (referred to Table 5).

Further, adsorption capacities were measured by measuring 8 day-release concentrations of chlorine dioxide-adsorbed 2 g carriers obtained from Experimental Example 2 to 13 in the same manner as in Experimental Example 1 (referred to Table 6).

### Comparative Examples 1 to 12

Experiments were conducted on respective composition ratios of Comparative Examples 1 to 12 by varying only the composition ratio of each mineral in the same manner as in Experimental Example 1 above (referred to Table 5).

Further, adsorption capacities were measured by measuring 8 day-release concentrations of chlorine dioxide-adsorbed 2 g carriers obtained from Comparative Example 1 to 12 in the same manner as in Experimental Example 1 (referred to Table 6).

### Composition Ratios of Minerals Forming the Carriers

TABLE 5

| Experimental | | | | | | Others |
|---|---|---|---|---|---|---|
| Example | | | | | | (TiO₂, K₂O, |
| (wt %) | SiO₂ | Al₂O₃ | Fe₂O₃ | MgO | CaO | SO₃) |
| Experimental | 56 | 13 | 8 | 8 | 4 | 11 |
| Example 1 | | | | | | |
| Experimental | 50 | 15 | 10 | 10 | 5 | 10 |
| Example 2 | | | | | | |
| Experimental | 62 | 10 | 7 | 7 | 4 | 10 |
| Example 3 | | | | | | |
| Experimental | 62 | 12 | 5 | 7 | 4 | 10 |
| Example 4 | | | | | | |
| Experimental | 62 | 12 | 7 | 5 | 4 | 10 |
| Example 5 | | | | | | |
| Experimental | 62 | 12 | 7 | 6 | 3 | 10 |
| Example 6 | | | | | | |
| Experimental | 62 | 12 | 7 | 7 | 4 | 8 |
| Example 7 | | | | | | |
| Experimental | 69 | 10 | 5 | 5 | 3 | 8 |
| Example 8 | | | | | | |
| Experimental | 57 | 15 | 7 | 7 | 4 | 10 |
| Example 9 | | | | | | |
| Experimental | 57 | 12 | 10 | 7 | 4 | 10 |
| Example 10 | | | | | | |
| Experimental | 57 | 11 | 8 | 10 | 4 | 10 |
| Example 11 | | | | | | |
| Experimental | 58 | 12 | 8 | 8 | 5 | 9 |
| Example 12 | | | | | | |
| Experimental | 57 | 11 | 8 | 8 | 4 | 12 |
| Example 13 | | | | | | |
| Comparative | 45 | 16 | 11 | 11 | 5 | 12 |
| Example 1 | | | | | | |
| Comparative | 60 | 8 | 9 | 9 | 4 | 10 |
| Example 2 | | | | | | |
| Comparative | 62 | 13 | 3 | 8 | 4 | 10 |
| Example 3 | | | | | | |
| Comparative | 64 | 12 | 7 | 3 | 4 | 10 |
| Example 4 | | | | | | |
| Comparative | 61 | 12 | 8 | 8 | 1 | 10 |
| Example 5 | | | | | | |
| Comparative | 63 | 13 | 7 | 7 | 4 | 6 |
| Example 6 | | | | | | |
| Comparative | 75 | 9 | 4 | 4 | 2 | 6 |
| Example 7 | | | | | | |
| Comparative | 55 | 17 | 7 | 7 | 4 | 10 |
| Example 8 | | | | | | |
| Comparative | 55 | 12 | 12 | 7 | 4 | 10 |
| Example 9 | | | | | | |
| Comparative | 55 | 12 | 8 | 12 | 4 | 9 |
| Example 10 | | | | | | |
| Comparative | 57 | 12 | 7 | 7 | 7 | 10 |
| Example 11 | | | | | | |
| Comparative | 56 | 12 | 7 | 7 | 4 | 14 |
| Example 12 | | | | | | |

### Results of Adsorption Capacity Experiments of the Carriers According to the Respective Composition Ratios

TABLE 6

| Experimental | | | | | | | | |
|---|---|---|---|---|---|---|---|---|
| Example | 1 | 2 | 3 | 4 | 5 | 6 | 7 | 8 |
| (ppm) | day | days | days | days | days | days | days | days |
| Experimental | 2.0 | 2.0 | 2.0 | 2.0 | 2.0 | 1.9 | 1.6 | 1.4 |
| Example 1 | | | | | | | | |
| Experimental | 2.0 | 2.0 | 2.0 | 2.0 | 2.0 | 1.6 | 1.3 | 1.2 |
| Example 2 | | | | | | | | |
| Experimental | 2.0 | 2.0 | 2.0 | 2.0 | 2.0 | 1.7 | 1.4 | 1.2 |
| Example 3 | | | | | | | | |
| Experimental | 2.0 | 2.0 | 2.0 | 2.0 | 2.0 | 1.9 | 1.7 | 1.5 |
| Example 4 | | | | | | | | |
| Experimental | 2.0 | 2.0 | 2.0 | 2.0 | 2.0 | 1.9 | 1.7 | 1.5 |
| Example 5 | | | | | | | | |
| Experimental | 2.0 | 2.0 | 2.0 | 2.0 | 2.0 | 1.9 | 1.7 | 1.5 |
| Example 6 | | | | | | | | |
| Experimental | 2.0 | 2.0 | 2.0 | 2.0 | 2.0 | 1.9 | 1.7 | 1.5 |
| Example 7 | | | | | | | | |
| Experimental | 2.0 | 2.0 | 2.0 | 2.0 | 2.0 | 1.7 | 1.5 | 1.2 |
| Example 8 | | | | | | | | |
| Experimental | 2.0 | 2.0 | 2.0 | 2.0 | 2.0 | 1.6 | 1.4 | 1.3 |
| Example 9 | | | | | | | | |
| Experimental | 2.0 | 2.0 | 2.0 | 2.0 | 2.0 | 1.9 | 1.8 | 1.5 |
| Example 10 | | | | | | | | |
| Experimental | 2.0 | 2.0 | 2.0 | 2.0 | 2.0 | 1.9 | 1.8 | 1.6 |
| Example 11 | | | | | | | | |
| Experimental | 2.0 | 2.0 | 2.0 | 2.0 | 2.0 | 1.8 | 1.6 | 1.5 |
| Example 12 | | | | | | | | |
| Experimental | 2.0 | 2.0 | 2.0 | 2.0 | 2.0 | 1.9 | 1.7 | 1.4 |
| Example 13 | | | | | | | | |
| Comparative | 2.0 | 2.0 | 1.8 | 1.6 | 1.3 | 1.1 | 0.9 | 0.5 |
| Example 1 | | | | | | | | |
| Comparative | 2.0 | 2.0 | 1.9 | 1.7 | 1.4 | 1.0 | 0.8 | 0.5 |
| Example 2 | | | | | | | | |
| Comparative | 2.0 | 2.0 | 2.0 | 2.0 | 1.9 | 1.5 | 1.2 | 1.0 |
| Example 3 | | | | | | | | |
| Comparative | 2.0 | 2.0 | 2.0 | 2.0 | 1.7 | 1.3 | 1.1 | 0.9 |
| Example 4 | | | | | | | | |
| Comparative | 2.0 | 2.0 | 2.0 | 2.0 | 1.7 | 1.2 | 1.0 | 0.8 |
| Example 5 | | | | | | | | |
| Comparative | 2.0 | 2.0 | 2.0 | 2.0 | 1.9 | 1.5 | 1.2 | 1.0 |
| Example 6 | | | | | | | | |
| Comparative | 2.0 | 2.0 | 1.8 | 1.6 | 1.3 | 1.1 | 0.8 | 0.4 |
| Example 7 | | | | | | | | |
| Comparative | 2.0 | 2.0 | 1.8 | 1.6 | 1.4 | 1.0 | 0.8 | 0.4 |
| Example 8 | | | | | | | | |
| Comparative | 2.0 | 2.0 | 2.0 | 1.9 | 1.7 | 1.4 | 1.2 | 1.0 |
| Example 9 | | | | | | | | |
| Comparative | 2.0 | 2.0 | 2.0 | 1.5 | 1.3 | 1.2 | 1.1 | 0.9 |
| Example 10 | | | | | | | | |
| Comparative | 2.0 | 2.0 | 2.0 | 1.6 | 1.4 | 1.2 | 1.0 | 0.9 |
| Example 11 | | | | | | | | |
| Comparative | 2.0 | 2.0 | 2.0 | 1.5 | 1.3 | 1.2 | 1.0 | 0.8 |
| Example 12 | | | | | | | | |

It may be confirmed that adsorption capacities show remarkably excellent release concentrations and duration times compared to those of a silica gel adsorbent in the related art (the invention 10-1443455) within the composition ratio range presented in the present disclosure through these Experimental Examples 1 to 13, and adsorption capacities did not show such remarkably excellent release concentrations and duration times outside the numerical value range presented in the present disclosure through Comparative Examples 1 to 12.

Specifically, Experimental Examples 2 to 7 evaluate adsorption capacities using the minimum composition ratio in the composition ratio range presented by the present disclosure as any one component with respect to the composition ratio of each mineral, and using randomly specified composition ratios in the numerical value range presented by the present disclosure with respect to composition ratios of the rest of the minerals.

Specifically, Experimental Examples 8 to 13 evaluate adsorption capacities using the maximum composition ratio in the composition ratio range presented by the present disclosure as any one component with respect to the composition ratio of each mineral, and using randomly specified composition ratios in the numerical value range presented by the present disclosure with respect to composition ratios of the rest of the minerals.

Further, Comparative Examples 1 to 6 are experimental results for showing that remarkable adsorption capabilities were not shown when using a numerical value range lower than the range presented by the present disclosure as the composition ratio of any one component with respect to the composition ratio of each mineral, and using specific numerical values in the numerical value range presented by the present disclosure as composition ratios of the rest of the minerals.

Further, Comparative Examples 7 to 12 are experimental results for showing that remarkable adsorption capabilities were not shown when using a numerical value range higher than the range presented by the present disclosure as the composition ratio of any one component with respect to the composition ratio of each mineral, and using specific numerical values in the numerical value range presented by the present disclosure as composition ratios of the rest of the minerals.

### Example 2

In order to stable release a chlorine dioxide gas for a long period of time, a chlorine dioxide release kit was prepared in the following manner.

After putting 400 g of a carrier adsorbed with a high concentration chlorine dioxide together with an indicator into a 500 ml capacity well-light shielded polyethylene sealed container, 20 g of a bead-type gel in which aromatic substances were immersed in a super absorbent polymer was put into an upper portion of the container. After forming a hole with a hole size of 1 mm to 3 mm in a sealed container lid to adjust the release amount, a chlorine dioxide release kit was prepared by closing the hole-formed lid.

After installing a Y-shaped connecting pipe at the exit of the hole as shown in FIGS. 6A and 6B in order to grasp the release concentration and the long-term concentration maintenance performance of the prepared kit, and allowing an air pump to pass air through the kit in a flow rate of 2 liters per minute, release concentrations were measured with a measuring instrument at room temperature at certain intervals over three months. As a result, it was confirmed that, although the concentration of a chlorine dioxide gas released to the outside of the sealed container for three months varied in the release amount and the duration time depending on the size of the hole, chlorine dioxide at a concentration of about 25 ppm was released constantly and continuously for three months when the hole size was 2 mm.

Further, to examine the applicability of the release kit as a sterilizing device in the future, the release kit of FIGS. 6A and 6B was installed so that chlorine dioxide was automatically diluted by connecting the outlet part of the Y-shaped connecting pipe of a chlorine dioxide release kit to the inlet of an air blower fan with the ventilation capacity of about 4,000 L per minute in an indoor space of 240 m³ (referred to FIGS. 6a and 6B). After measuring the concentration of chlorine dioxide finally released from the ventilator outlet at regular intervals for three months, it was confirmed that the chlorine dioxide concentration of a 0.01 ppm level, which had been about 2,000 times diluted, was continuously released. It could be seen based on these results that the chlorine dioxide release kit might be safely used for sterilization at a level of 1/10 of 0.1 ppm, i.e., the allowable safety concentration for human body, in indoor spaces in multiuse facilities, examining rooms in the hospital, etc. requiring sterilization of bacteria and viruses. Further, it was confirmed that the hole size was adjustable depending on the sterilization purposes at a higher concentration (0.1 ppm or less) by enlarging the size of the hole if necessary.

What is claimed is:

1. A chlorine dioxide release kit including:
a carrier onto which a chlorine dioxide gas is adsorbed;
a sealed container; and
a lid, wherein the carrier onto which the chlorine dioxide gas is adsorbed is prepared by mixing a powder having composition ratios of 50 wt % to 69 wt % of SiO₂, 10 wt % to 15 wt % of Al₂O₃, 5 wt % to 10 wt % of Fe₂O₃, 5 wt % to 10 wt % of MgO, 3 wt % to 5 wt % of CaO, and 4 wt % to 8 wt % of others including TiO₂, K₂O, and SO₃ with an activated carbon powder.

2. The chlorine dioxide release kit according to claim 1, wherein the activated carbon powder has a composition ratio of 10 wt % to 50 wt %.

3. The chlorine dioxide release kit according to claim 1, wherein the activated carbon powder has a composition ratio of 15 wt % to 25 wt %.

4. The chlorine dioxide release kit according to claim 1, wherein the carrier onto which the chlorine dioxide gas is adsorbed is made of spherical beads having an average size of 2 mm to 3 mm.

5. The chlorine dioxide release kit according to claim 1, wherein the carrier onto which the chlorine dioxide gas is adsorbed has a specific surface area (BET) distribution of 70 m²/g to 150 m²/g.

6. The chlorine dioxide release kit according to claim 1, wherein the lid has a hole with a diameter of 1 mm to 5 mm to determine a release amount and a duration time.

7. The chlorine dioxide release kit according to claim 1, wherein the lid has a hole with a diameter of 1 mm to 3 mm to determine a release amount and a duration time.

8. The chlorine dioxide release kit according to claim 1, further including a polyamide air freshener gel for preventing a unique smell of chlorine dioxide from being generated from the chlorine dioxide release kit.

9. The chlorine dioxide release kit according to claim 1, wherein the carrier onto which the chlorine dioxide gas is adsorbed further includes an indicator material which indicates whether or not the chlorine dioxide gas is adsorbed or desorbed, by colors.

10. The chlorine dioxide release kit according to claim 9, wherein the indicator material is a silica gel.

11. A preparation apparatus capable of preparing a carrier onto which a chlorine dioxide gas is adsorbed of claim 1, the preparation apparatus comprising:
a reaction tank capable of producing aqueous chlorine dioxide by dissolving a solid NaClO₂ powder, i.e., a raw material of chlorine dioxide in water, and adding acid to the solid NaClO₂ powder-dissolved water;
a gas pump capable of discharging a chlorine dioxide gas to the outside by injecting air into the reaction tank in a flow amount of 2 L/min or more;
an adsorption bed capable of filling the carrier and adsorbing the chlorine dioxide gas; and
an indicator capable of indicating an adsorption amount and an adsorption progress degree of the chlorine dioxide gas by colors.

12. The preparation apparatus according to claim 11, wherein the indicator is a silica gel.

13. The preparation apparatus according to claim 11, further including one or more among a stirrer, a thermometer, a pressure gauge, a flowmeter, a cryostat, and a granulated activated carbon bed capable of removing the portion of the chlorine dioxide gas when a portion of the chlorine dioxide gas is discharged to the outside after the adsorption process is completed.
